(19) United States
(12) Patent Application Publication
(10) Pub. No.: US 2010/0217073 A1
(43) Pub. Date: Aug. 26, 2010

(54) ELECTROSURGICAL INSTRUMENT, AND AN ENDOSCOPE WITH A CORRESPONDING INSTRUMENT

(76) Inventors: Klaus Fischer, Nagold (DE); Matthias Voigtländer, Gomaringen (DE); Daniel Schäller, Tubingen (DE); Mara Szyrach, Tubingen (DE); Lars Blobel, Ammerbuch-Entringen (DE); Irina Sigle, Mossingen (DE)

Correspondence Address: DICKSTEIN SHAPIRO LLP, 1825 EYE STREET NW, Washington, DC, 20006-5403, US

(21) Appl. No.: 12/675,468

(22) PCT Filed: Jul. 31, 2008
(86) PCT No.: PCT/EP2008/006330

§ 371 Date: Feb. 26, 2010

(30) Foreign Application Priority Data
Aug. 29, 2007 (DE) 10 2007 040 842.2

(51) Int. Cl. A61B 1/018; A61B 18/14

(52) U.S. Cl. 600/104; 606/41

(57) ABSTRACT

An electrosurgical instrument including a protective tube, to be inserted into a working channel of an endoscope, and an electrode disposed within the protective tube. The electrode is movable in a distal direction and a proximal direction inside the protective tube by a control element. By being moved in the distal direction, the electrode can be brought into a deployed position in which the electrode at least partially protrudes from a distal end of the protective tube. The distal end of the protective tube has a closure element with an opening for the passage of the electrode. The opening of the closure element has a maximum internal diameter smaller than the internal diameter of the protective tube and is used for guiding and/or cleaning the electrode.

### FIELD OF THE DISCLOSED EMBODIMENTS

The invention relates to an electrosurgical instrument, and particularly to an electrosurgical instrument that is easily guidable and cleanable while in use, and an endoscope with a corresponding instrument.

### BACKGROUND

EP 1 293 169 B1 discloses an endoscope. It includes at least one working channel into which a protective tube may be inserted. This protective tube is used for guiding instruments. In an endoscopic submucosal dissection (ESD), the endoscope could accommodate a needle-shaped cutting instrument. EP 1 293 169 B1 illustrates an electrosurgical instrument for coagulation. Instruments are also known for dissection or resection wherein tissue is cut precisely and coagulated as necessary by means of high-frequency surgery.

In a typical ESD, fluid is injected into the submucosa to elevate the mucosa from the muscularis and to obtain a sufficiently large hollow space for resection. Needle-shaped cutting instruments are preferably used in an ESD.

In endoscopic dissections, particularly using needle-shaped instruments, determining and adjusting the position of the instrument's tip relative to the protective tube but also relative to the end of the working channel presents a major problem. If the position is determined incorrectly or if it is poorly adjusted, this may result in injuries or unintentional perforations, particularly of the muscularis.

It is sometimes helpful for easy handling of the instrument if various, predefined positions of the cutting instrument are provided relative to the protective tube. In the ESD, for example, one needs a needle length that is as short as possible for placing markers and a needle length that is as long as possible for circumcision.

In practice, precise determination and adjustment of the position of the cutting instrument, for example an active cutting electrode, depends greatly on the visual control of the doctor performing the operation.

This method of precise presetting or provision of specific, previously defined positions fails due to numerous problems. For one, not only is it necessary to allow for the position of the instrument's tip relative to the protective tube but also for the position of the protective tube's tip relative to the working channel. Depending on the material used, the protective tube may be stretched when force is applied, thereby altering the length of the tube and, consequently, the position of the instrument's tip. Furthermore, the constant movement of the flexible working channel and the movement of the protective tube inside the working channel may alter the position of an instrument previously positioned inside the protective tube.

Added to this is that, even with safe determination of the instrument's position relative to the protective tube, the protective tube protrudes into the tissue due to slight pressure and thus changes the maximum cutting depth achievable. Therefore, it should also be possible to determine precisely the position of the protective tube's distal end.

In the case of a visual check, this difficulty is compounded by the fact that the distal end of the surgical instrument is frequently not visible because of contamination due to tissue residues and further use of the device is at the very least made more difficult. In order to remove these tissue residues, there is sometimes no other option than to take the instrument out of the working channel with or without the protective tube. The instrument is then frequently replaced with a new, sterile instrument. The costs for this are not inconsiderable.

### SUMMARY

Thus, the object of the disclosed embodiments is to provide an electrosurgical instrument that can be managed easily and safely.

Disclosed embodiments include an electrosurgical instrument including a protective tube insertable in a working channel of an endoscope and an electrode which is movable in a distal direction and a proximal direction inside the protective tube by means of a control element, wherein the electrode, by being moved in the distal direction, can be brought into a deployed position in which the electrode at least partially protrudes from a distal end of the protective tube. The distal end of the protective tube includes a closure element with an opening for the passage of the electrode, wherein the opening of the closure element, for guiding and/or cleaning the electrode, has a maximum internal diameter d1 smaller than the internal diameter d2 of the protective tube.

The electrode or the cutting instrument may thus be moved backwards and forwards inside the protective tube by a control element. To achieve a deployed position of the electrode, it is moved in the distal direction (relative to the protective tube) until at least a section of the electrode protrudes from the distal end of the protective tube.

The distal end of the protective tube has a narrowing with an opening through which the electrode is guided to reach the deployed position. The opening is used in particular for guiding and/or cleaning the electrode. Preferably, said opening has a maximum internal diameter smaller than the internal diameter of the protective tube. If the opening is circular in shape, the maximum internal diameter of the opening is identical to the diameter of the electrode. Alternatively, the opening may have any expedient shape. For example, it may be elliptical, square, rectangular, etc. For the disclosed embodiments, it is merely necessary for the opening of the closure element to restrict the interior of the tube in cross-section.

In one disclosed embodiment, the electrode includes a stopper element which cooperates with the closure element such that it restricts the movement of the electrode in the distal direction. Thus, together with the closure element, the stopper element represents a mechanical restriction of the electrode's movement. As a result, this restricts the electrode's deployed length in the distal direction.

In one disclosed embodiment, the stopper element is shaped cylindrically. Thus, for example, the stopper element is shaped according to the interior of the protective tube and is movable backwards and forwards inside it. This design of the stopper element is particularly advantageous if the electrode's cross-section is substantially smaller than that of the protective tube. The stopper element then enables the electrode to be guided without force and/or specifically into the opening of the closure element. Mechanical restriction of the electrode's movement may be ensured in a simple manner by the stopper element coming into contact at least in sections with portions of the closure element.

In one disclosed embodiment, a spring element, in particular a coil spring, is disposed between the stopper element and the closure element whereby the spring element injects a force in the proximal direction in the deployed position of the electrode. So by moving the electrode, by means of the control element for example, in the distal direction, a spring force is created that counteracts this movement. The spring element is used to provide an automatic retraction mechanism of the electrode into a region inside the protective tube. It is thus possible to prevent unintentional cutting of the tissue. The spring force also restricts the electrode's ability to move in the distal direction.

In one disclosed embodiment, the opening of the electrode's closure element is designed according to the shape of the electrode's cross-section. Thus the opening is adapted in cross-section to the electrode's cross-section such that, particularly on guiding the electrode from the deployed position to back inside the protective tube, the electrode is wiped of any deposits adhering to it, in particular any tissue deposits. This enables the operative intervention to be carried out substantially more efficiently since it is unnecessary to replace the electrode with its associated control elements. Due to this simple cleaning process, it is also possible to improve visual control when guiding the electrode.

In one disclosed embodiment, the opening of the closure element is designed for feeding the electrode through without force.

In one disclosed embodiment, the electrode may include a needle. Such a development of the instrument is advantageous for an ESD.

In particular, it is possible for this application to provide a capillary line inside the electrode for injecting fluid. For example, in an ESD fluid can be injected via the capillary line into the submucosa to elevate it from the muscularis. It is possible to dispense with time-consuming changing of the instrument during the operation.

In one disclosed embodiment, the protective tube includes at least one marker on the outside of the protective tube close to its distal end. In particular, this marker may be ring-shaped. These markers may be disposed at a predefined distance from the protective tube's distal tip and make it easier to visually determine the protective tube's position, in particular the position of its end. In particular, it may be ascertained how far the protective tube's end protrudes onto or into tissue to be treated.

In one disclosed embodiment, a fixing element is formed on the protective tube's distal end such that the fixing element induces a defined holding force that counteracts any movement of the electrode in the distal and/or proximal direction. The fixing element may either attach directly to the electrode or may act on it by way of the control element. The electrode is fixed in relation to the protective tube by means of the fixing element in such a manner that unintentional displacement of the electrode relative to the protective tube is prevented. Thus it is easily possible to maintain a previously set position of the electrode relative to the protective tube's distal end.

Preferably, the holding force that is induced is designed to be slightly higher than the friction or adhesion forces of the protective tube and endoscope. As a result, the protective tube may be moved easily inside the endoscope's working channel without any change occurring in the endoscope's position relative to the distal end of the protective tube.

In a further disclosed embodiment, the fixing element includes at least one elastic element that is disposed inside the protective tube. This elastic element can induce the holding force between the electrode or control element and the protective tube.

In a further disclosed embodiment, the electrosurgical element includes a control unit for adjusting the position of the electrode relative to the protective tube's distal end. Thus the control unit makes it easier to adjust the relative position.

Preferably, the control unit includes at least one locking device with which the control element and thus the electrode can be fixed in at least one setting position. Thus, once set, it is easily possible to maintain a position of the electrode relative to the protective tube's distal end.

In a further disclosed embodiment, the control unit has at least two setting positions whereby in a first setting position the electrode at least partially protrudes from a distal end of the protective tube and in a second setting position the electrode lies inside the protective tube. Such previously defined setting positions can make handling of the electrosurgical instrument considerably easier. It is possible to guarantee safe adjustment of the relative positions even where there is little visible contact.

A further embodiment includes an endoscope with at least one working channel and with an electrosurgical instrument such as has previously been described. The advantages of such an endoscope emerge in a similar manner as has already been described with respect to the electrosurgical instrument.

### BRIEF DESCRIPTION OF THE DRAWINGS

The disclosed embodiments will be described in the following on the basis of example embodiments which will be explained in greater detail with reference to the enclosed drawings.

FIG. 1 is a schematic cross-section through an endoscope.

FIG. 2 is a needle-shaped instrument with protective tube in an ESD.

FIG. 3 is a cross-section through the distal end of an electrosurgical instrument according to a disclosed embodiment.

FIG. 4 is another cross-section through the distal end of the electrosurgical instrument of FIG. 3.

FIG. 5 is a cross-section through the distal end of an electrosurgical instrument with a deployed electrode, according to another disclosed embodiment.

FIG. 6 is a cross-section through the distal end of the electrosurgical instrument of FIG. 5, with a retracted electrode.

FIG. 7 is a cross-section through the distal end of an electrosurgical instrument with an elastomer ring, according to another disclosed embodiment.

FIG. 8 is another cross-section through the distal end of the electrosurgical instrument of FIG. 7.

FIG. 9 is a cross-section through a needle electrode according to a disclosed embodiment.

FIG. 10 is a schematic diagram of a partial section of an electrosurgical instrument according to the disclosed embodiments, being used in an ESD.

FIG. 11 is another schematic diagram of a partial section of an electrosurgical instrument according to the disclosed embodiments, being used in an ESD.

FIG. 12 is a schematic endoscopic view onto the electrosurgical instrument according to the disclosed embodiments.

FIG. 13 is a control unit for an electrosurgical instrument according to disclosed embodiments.

FIG. 14 is another view of the control unit for an electrosurgical instrument of FIG. 13.

### DETAILED DESCRIPTION

The same reference numerals are used in the following description for identical parts and parts acting in an identical manner.

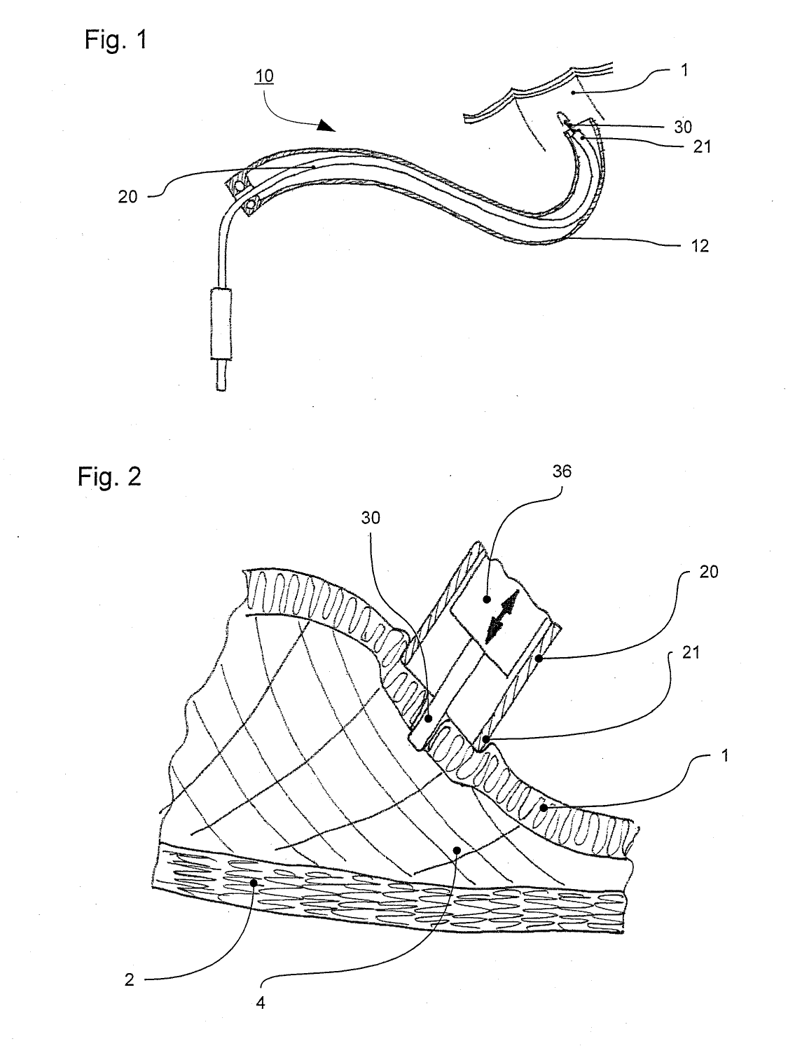

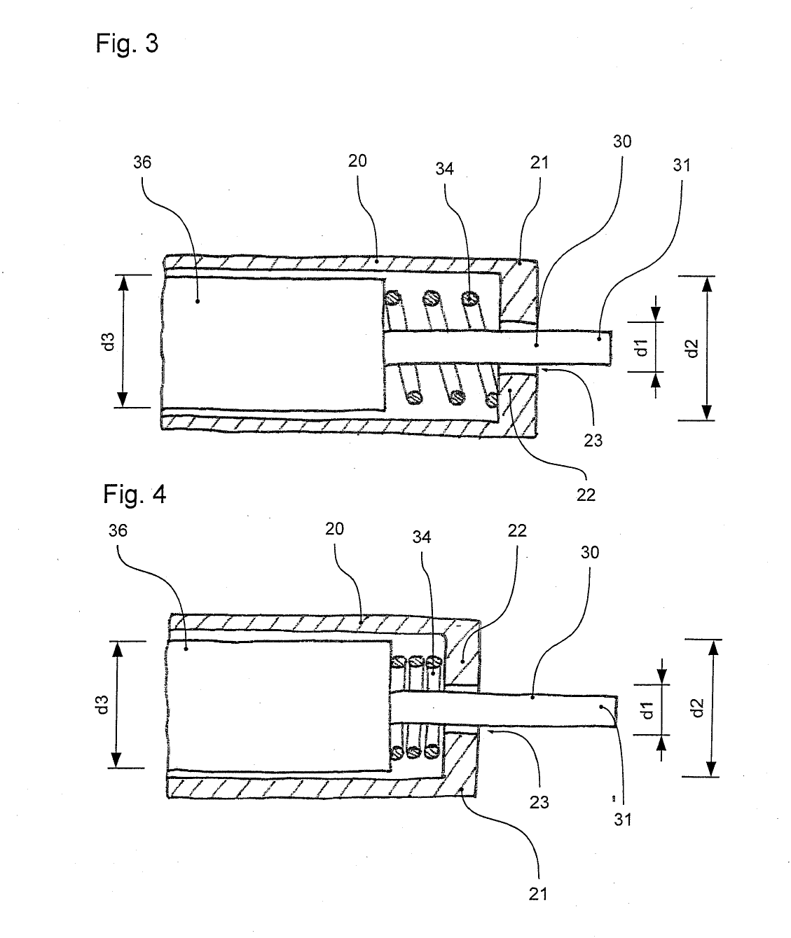

FIG. 1 illustrates a highly schematic cross-section through an endoscope 10. Endoscope 10 includes a working channel 12 into which a protective tube 20 is introduced. Inside protective tube 20 is a guiding device 36 (FIG. 2) that is in operative connection with a needle electrode 30. By means of guiding device 36, needle electrode 30 may be moved in the distal and proximal direction inside protective tube 20. In the endoscope 10 of FIG. 1, needle electrode 30 is in a deployed position in which tip 31 (cf. FIGS. 3 and 4) of needle electrode 30 protrudes from distal end 21 of protective tube 20.

FIG. 1 also illustrates in schematic form the outside wall of a hollow organ, in particular mucosa 1 thereof, into which needle electrode 30 is to be inserted.

As can be seen in FIG. 1, determining the position of needle electrode 30 is complex since both working channel 12 and protective tube 20 are independently flexible. A strong curvature thereof not only changes the friction properties of the device but can also lead to a change in the absolute position of needle electrode 30 or its position relative to working channel 12 and/or to protective tube 20.

FIG. 2 shows a hollow organ in cross-section. During an ESD, fluid 4 has been introduced via needle electrode 30 into mucosa 1 and the mucosa 1 has thus been separated from muscularis 2 of the hollow organ. Fluid 4 creates an intermediate space which ensures a sufficiently large gap between the two layers during resection.

As can be seen in FIG. 2, visual determination of the position of the distal end 21 of the protective tube 20 and/or electrode 30 is difficult and sometimes impossible when using conventional endoscopes 10. Since protective tube 20 of endoscope 10 may partly penetrate into the soft tissue of mucosa 1 (as in the example shown in FIG. 2), distal end 21 of protective tube 20 may become concealed by the tissue and the folds that form. Therefore it is not possible to visually identify distal end 21. Determining the position of needle electrode 30, which can be moved in the distal and proximal direction by a guiding device 36, is correspondingly problematic. In such an arrangement, it is not possible to determine the absolute position visually even when the position of needle electrode 30 relative to protective tube 20 is known.

FIG. 3 shows distal end 21 of a protective tube 20 according to a disclosed embodiment. Protective tube 20 includes a closure element 22 that extends from distal end 21 of protective tube 20, essentially perpendicular to the outer skin of protective tube 20, into its interior. Closure element 22 forms an opening 23 having a maximum internal diameter d1 that is considerably smaller than internal diameter d2 of protective tube 20. Thus distal end 21 of the protective tube is narrowed. Opening 23 extends parallel to the longitudinal direction of a longish needle electrode 30 and is suitable for passing this electrode through without friction.

Needle electrode 30 is placed centrally on a guiding device 36 which is also located inside protective tube 20 and enables needle electrode 30 to be moved in the distal and proximal direction as a result of which the position of tip 31 of needle electrode 30 changes relative to protective tube 20, in particular distal end 21 thereof.

Guiding device 36 is formed in a cylindrical shape and has an internal diameter d3 that is larger than internal diameter d1 of opening 23 and smaller than internal diameter d2 of protective tube 20. Thus, guiding device 36 may be moved inside protective tube 20 with little application of force. Internal diameter d3 of guiding device 36 and attachment of needle electrode 30 thereto is designed such that it is easily possible to insert needle electrode 30 into opening 23. Due to the difference between internal diameter dl of opening 23 and internal diameter d3 of guiding device 36, closure element 22 together with cylindrical guiding device 36 forms a stopper element which mechanically restricts the movement of guiding device 36 in the distal direction.

Disposed between closure element 22 and guiding device 36 is a spring element 34. In the embodiment shown in FIGS. 3 and 4, this spring element 34 is a coil spring which encloses needle electrode 30 in sections. As needle electrode 30 and guiding device 36 are moved in the distal direction, the spring element 34 becomes more compressed, resulting in the build-up of a force that counteracts the direction of movement. As soon as the doctor who is operating stops exerting force on guiding device 36, needle electrode 30 is retracted due to the force induced by spring element 34. It is possible to differentiate, for example, between three positions of needle electrode 30 or of the corresponding needle length (distance between distal end 21 of protective tube 20 and tip 31 of needle electrode 30):
Needle length=0 when no force is applied via guiding device 36;
Short needle length with slight application of force (cf. FIG. 3);
Maximum needle length with great application of force (cf. FIG. 4).

The force induced by spring element 34 helps the doctor to operate needle electrode 30 and to determine its length. If the doctor is familiar with the instrument, he can estimate how far tip 31 protrudes from distal end 21 of protective tube 20 based on the force necessary to operate needle electrode 30.

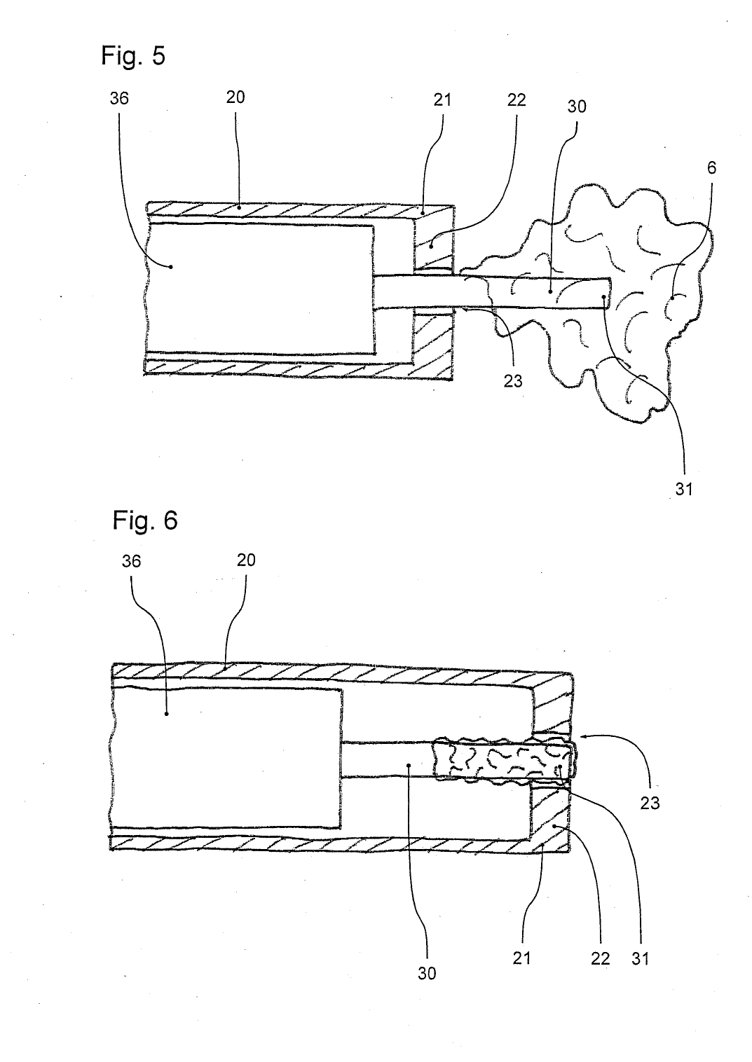

FIGS. 5 and 6 illustrate a further advantage of closure element 22. Here too, distal end 21 of protective tube 20, needle electrode 30 and guiding device 36 are illustrated. Illustration of spring element 34 has been omitted.

Due to the small difference between internal diameter d1 of opening 23 and the internal diameter of cylindrically formed needle electrode 30, the closure element may be used for cleaning needle electrode 30. Tissue 6, which adheres to the side walls of needle electrode 30 during the operation, can be wiped off on closure element 22 when needle electrode 30 is retracted into the interior of protective tube 20. On redeploying needle electrode 30, its tip 31 is easily identifiable. This enables effective visual control of the position of needle electrode 30.

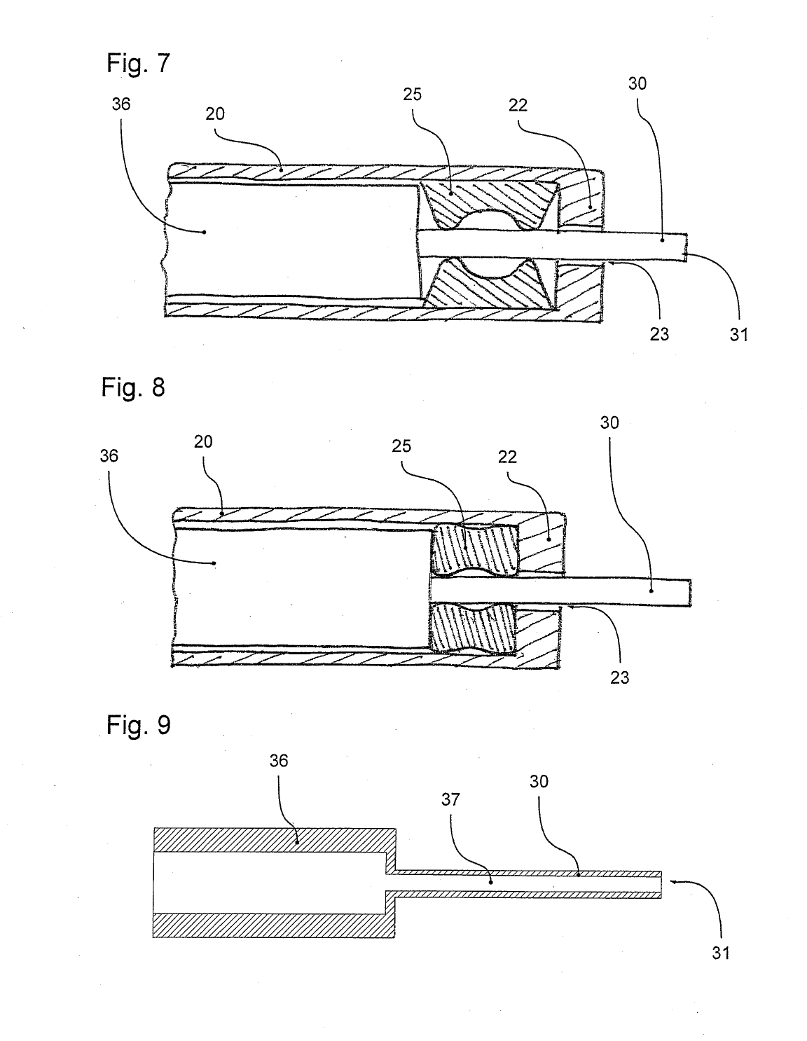

FIGS. 7 and 8 show a further embodiment of protective tube 20 with corresponding guiding device 36 and needle electrode 30. An intermediate space between closure element 22 and guiding device 36 is filled by an elastomer ring 25. This elastomer ring 25 surrounds needle electrode 30 and is firmly connected to protective tube 20. Elastomer ring 25 is formed in such a manner that it induces a defined adhesive force which fixes needle electrode 30 in the previously set position. Elastomer ring 25 thus forms a fixing element which encompasses needle electrode 30 on distal end 21 of protective tube 20. Elastomer ring 25 may be designed in such a manner that the corresponding position of needle electrode 30 is maintained even if tube 20 or working channel 12 is severely bent, and if there are correspondingly strong movements. The fixing element thus induces an adhesive force which must be overcome during a movement of tip 31 of the needle electrode relative to protective tube 20. FIG. 8 shows needle electrode 30 in a position with maximum needle length, FIG. 7 in a position with short needle length. Elastomer ring 25 may also take over the function of a stopper element and/or that of a spring element 34 and/or of closure element 22. Furthermore, elastomer ring 25 may be designed in such a manner that introduction of needle electrode 31 into opening 23 is made easier. It is also possible to combine elastomer ring 25 of FIGS. 7 and 8 with the coil spring of FIGS. 3 and 4.

Needle electrode 30 preferably includes, as illustrated in FIG. 9, a capillary line 37 which enables the injection of fluid into sections of tissue by means of needle electrode 30. Thus needle electrode 30 can assume a dual function in an ESD. On one hand it acts as an injection needle for the introduction of fluid 4 into the tissue, and on the other as an electrode for cutting and coagulating the tissue.

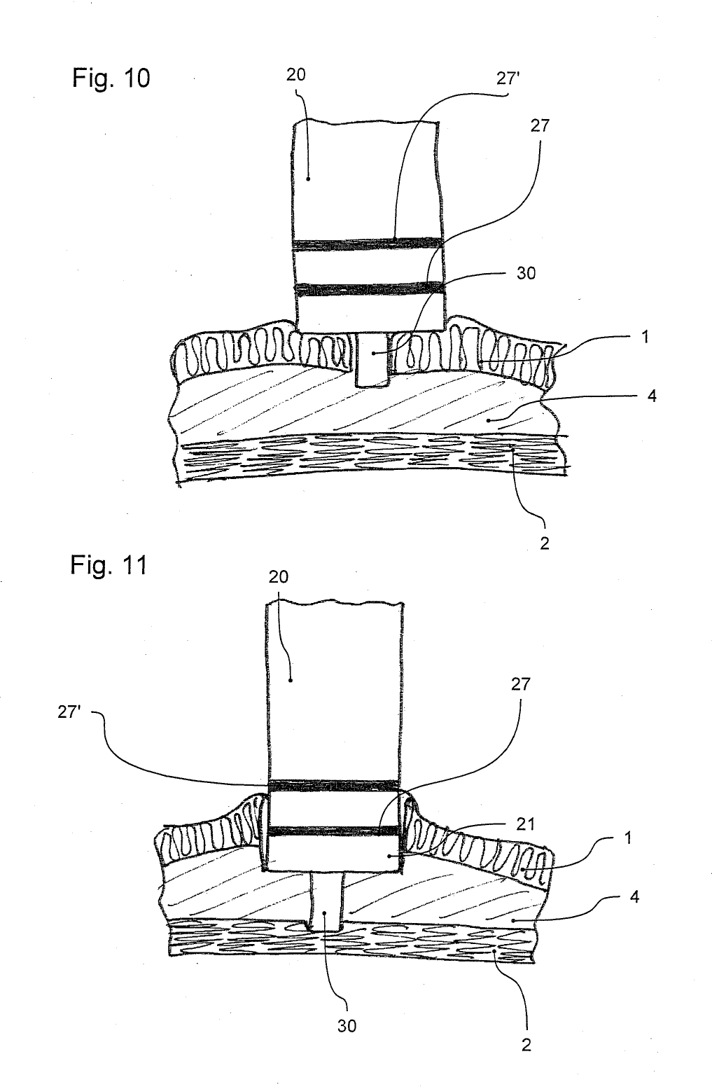

FIGS. 10 and 11 show a view from above onto protective tube 20, wherein needle electrode 30 protrudes at least partially from distal end 21 of protective tube 20. The instrument illustrated in FIGS. 10 and 11 is used to treat a hollow organ.

For this purpose, fluid 4 was introduced into an intermediate space between mucosa 1 and muscularis 2 for an ESD. Markers 27, 27′ on the outer wall of protective tube 20 close to the distal end help to determine the position of protective tube 20 relative to the tissue layers of the hollow organ. Markers 27, 27′ extend around protective tube 20 and have a defined distance to distal end 21 of the protective tube. Markers 27, 27′ are designed in such a manner that they can easily be identified visually. Markers 27, 27′ help to prevent excessively deep penetration of protective tube 20 into the tissue layers of the hollow organ (cf. FIG. 11). Alternatively, it is possible to determine the penetration depth of distal end 21 of protective tube 20 using markers 27, 27′ and to adapt the needle length accordingly.

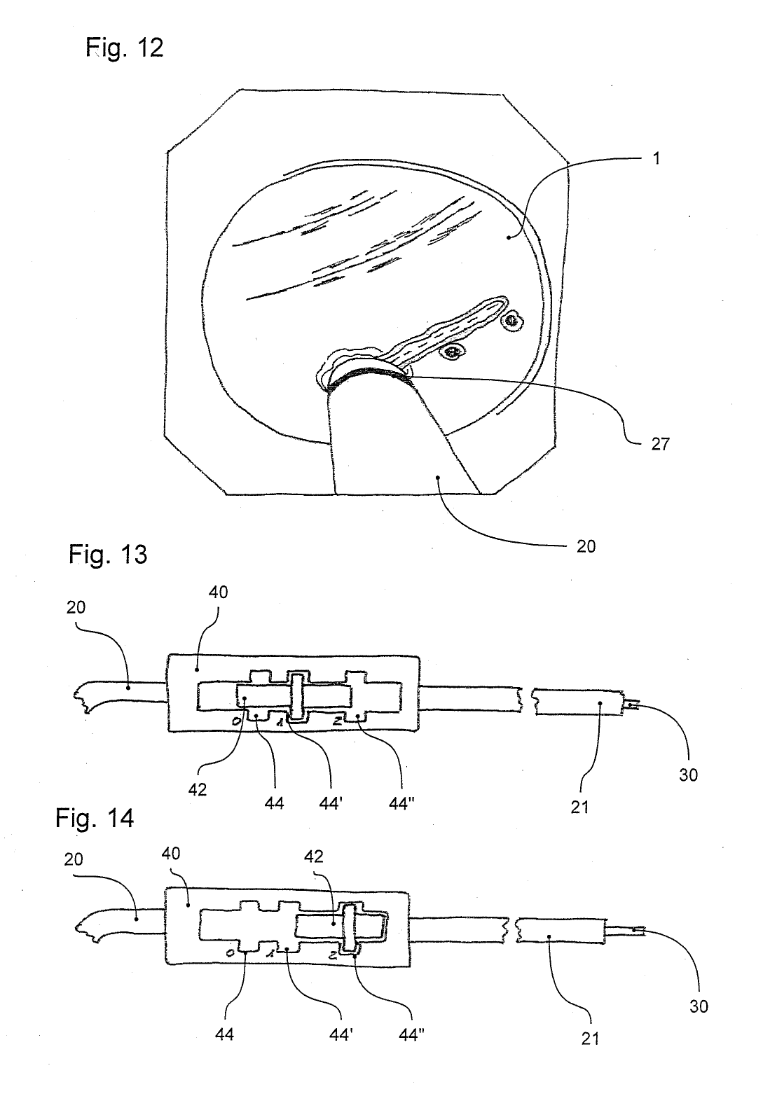

FIG. 12 represents an endoscopic image of protective tube 20. Estimation of depth, which is frequently difficult in images typical of endoscopes, is made easier by marker 27. In FIG. 12, distal end 21 of protective tube 20 rests on mucosa 1. Cutting of the tissue is ensured by needle electrode 30 which is not visible in the endoscopic diagram.

FIGS. 13 and 14 show a control unit 40 according to disclosed embodiments for adjusting the needle length of needle electrode 30. Control unit 40 is located on the proximal end of protective tube 20 and is easily accessible for the doctor.

Control unit 40 includes a slider 42 and a plurality of notches 44, 44′, 44″. Slider 42 is in operative connection with guiding device 36 (cf. FIGS. 3-6) and thus indirectly determines the position of needle electrode 30. Displacement of slider 42 in the distal direction leads to a movement of needle electrode 30 in the distal direction (cf. FIG. 14), a movement of slider 42 in the proximal direction leads to a movement of needle electrode 30 in the proximal direction (cf. FIG. 13). Slider 42 cooperates with notches 44, 44′, 44″ such that needle electrode 30 can be fixed in predefined positions. For this, a protrusion of slider 42 engages in either a first notch 44, a second notch 44′ or a third notch 44″. Control unit 40 is preferably developed in such a manner that the position of slider 42 in first notch 44 (cf. marking 0), second notch 44′ (cf. marking 1) or third notch 44″ (cf. marking 2) corresponds to a retracted position of needle electrode 30 into protective tube 20, or to a partially deployed position of needle electrode 30, or to a maximally deployed position of needle electrode 30. Thus the doctor need not rely on visual control of the needle position but may match his knowledge to that of the settings predefined by control unit 40. It is possible and desirable to use control unit 40 according to FIGS. 13 and 14 in combination with previously described protective tube 20 according to the disclosed embodiments.

It should be pointed out here that all the above described parts and in particular the details illustrated in the drawings are essential for the disclosed embodiments alone and in combination. Adaptations thereof are the common practice of persons skilled in the art.

1-15. (canceled)

16. An electrosurgical instrument comprising:
a protective tube; and
an electrode, the electrode being movable in a distal and proximal direction inside the protective tube,
wherein the electrode, by being moved in the distal direction, can be brought into a deployed position in which the electrode at least partially protrudes from a distal end of the protective tube,
wherein the distal end of the protective tube comprises a closure element with an opening for the passage of the electrode, and
wherein the opening of the closure element is sized relative to the electrode such that tissue residues are wiped off on retracting the electrode through the opening.

17. The electrosurgical instrument according to claim 16, further comprising a control element for controlling the movement of the electrode inside the protective tube.

18. The electrosurgical instrument according to claim 16, wherein the opening of the closure element has an internal diameter which is designed in relation to a diameter of the electrode to cause the tissue residues to be wiped off on retracting the electrode through the opening.

19. The electrosurgical instrument according to claim 16, wherein the opening of the closure element has a cross-section which is designed in relation to a cross-section of the electrode to cause the tissue residues to be wiped off on retracting the electrode through the opening.

20. The electrosurgical instrument according to claim 16, wherein the electrode comprises a stopper element which cooperates with the closure element such that it restricts the movement of the electrode in the distal direction.

21. The electrosurgical instrument according to claim 20, wherein the stopper element is formed cylindrically for guiding the electrode inside the protective tube without force.

22. The electrosurgical instrument according to claim 20, further comprising a spring element disposed between the stopper element and the closure element, said spring element inducing a force in the proximal direction in the deployed position of the electrode.

23. The electrosurgical instrument according to claim 22, wherein the spring element is a coil spring.

24. The electrosurgical instrument according to claim 16, wherein the opening of the closure element is designed according to the shape of the cross-section of the electrode.

25. The electrosurgical instrument according to claim 16, wherein the opening of the closure element is designed for feeding the electrode through without force.

26. The electrosurgical instrument according to claim 16, wherein the electrode comprises a needle.

27. The electrosurgical instrument according to claim 16, wherein the electrode comprises a capillary line for injecting fluid.

28. The electrosurgical instrument according to claim 16, wherein the protective tube comprises at least one ring-shaped marker on the outside of the protective tube near its distal end.

29. The electrosurgical instrument according to claim 16, further comprising a fixing element formed on the distal end of the protective tube such that the fixing element induces a defined holding force that counteracts any movement of the electrode in the distal and/or proximal direction.

30. The electrosurgical instrument according to claim 29, wherein the fixing element further comprises at least one elastic element that is disposed inside the protective tube.

31. The electrosurgical instrument according to claim 16, further comprising a control unit for adjusting the position of the electrode relative to the distal end of the protective tube.

32. The electrosurgical instrument according to claim 31, wherein the control unit comprises at least one locking device with which the control element can be fixed in at least one setting position.

33. The electrosurgical instrument according to claim 32, further comprising at least two setting positions, whereby in a first setting position the electrode at least partially protrudes from a distal end of the protective tube and in a second setting position the electrode lies inside the protective tube.

34. The electrosurgical instrument according to claim 16, wherein the protective tube is insertable into a working channel of an endoscope.

35. An endoscope comprising:
at least one working channel; and an electrosurgical instrument, wherein the electrosurgical instrument comprises:
a protective tube; and
an electrode, the electrode being movable in a distal and proximal direction inside the protective tube,
wherein the electrode, by being moved in the distal direction, can be brought into a deployed position in which the electrode at least partially protrudes from a distal end of the protective tube,
wherein the distal end of the protective tube comprises a closure element with an opening for the passage of the electrode, and
wherein the opening of the closure element is sized relative to the electrode such that tissue residues are wiped off on retracting the electrode through the opening.
